(19) United States
(12) Patent Application Publication
(10) Pub. No.: US 2021/0316681 A1
(43) Pub. Date: Oct. 14, 2021

(54) VEHICLE, CONTROL DEVICE, AND PROGRAM

(71) Applicant: TOYOTA JIDOSHA KABUSHIKI KAISHA, Aichi-ken (JP)

(72) Inventor: Miyako SAKAI, Toyota-shi (JP)

(21) Appl. No.: 17/175,701

(22) Filed: Feb. 15, 2021

(30) Foreign Application Priority Data
Apr. 8, 2020 (JP) 2020-069664

(51) Int. Cl. B60R 16/037; B60W 40/08; B60W 40/10; A61M 21/02

(52) U.S. Cl. CPC B60R 16/037; B60W 40/08; A61M 2021/0016; A61M 21/02; B60W 2510/1005; B60W 40/10

(57) ABSTRACT

A vehicle includes a scent discharge device configured to discharge a scent into a vehicle cabin. When a shift position is a travel position, the scent discharge device is controlled such that a first scent is discharged into the vehicle cabin, and when the shift position is a non-travel position, the control on the scent discharge device is changed from the control performed when the shift position is the travel position to a different control. Hereby, when the shift position is the travel position, the first scent is discharged. This makes is possible to restrain drowsiness of a driver while the driver is driving.

### CROSS-REFERENCE TO RELATED APPLICATION

This application claims priority to Japanese Patent Application No. 2020-069664 filed on Apr. 8, 2020, incorporated herein by reference in its entirety.

### BACKGROUND

### 1. Technical Field

The present disclosure relates to a vehicle, a control device, and a program.

### 2. Description of Related Art

In the related art, as a vehicle of this type, there has been proposed a vehicle including a drowsiness prevention device including a camera and an air gun (e.g., see Japanese Unexamined Patent Application Publication No. 2007-234060 (JP 2007-234060 A)). In the vehicle, it is determined whether or not a driver is dozing off based on a face image signal from the camera, and when the driver is dozing off, air including scent particles of awakening perfume is discharged toward the face of the driver from the air gun.

### SUMMARY

The above vehicle can restrain the driver from dozing off continuously because the vehicle awakes the driver when the driver is dozing off. However, the vehicle cannot sufficiently restrain the driver from starting dozing.

A main object of a vehicle, a control device, and a program of the present disclosure is to restrain drowsiness of a driver while the driver drives.

In order to achieve the main object, the vehicle, the control device, and the program of the present disclosure employ the following approach.

A vehicle according to the present disclosure is a vehicle including a scent discharge device and a control device. The scent discharge device is configured to discharge a scent into a vehicle cabin. The control device is configured to control the scent discharge device. When a shift position is a travel position, the control device controls the scent discharge device such that a first scent is discharged into the vehicle cabin, and when the shift position is a non-travel position, the control device changes the control on the scent discharge device from the control performed when the shift position is the travel position to a different control.

In the vehicle of the present disclosure, when the shift position is the travel position, the scent discharge device is controlled such that the first scent is discharged into the vehicle cabin, and when the shift position is the non-travel position, the control on the scent discharge device is changed from the control performed when the shift position is the travel position to a different control. Hereby, when the shift position is the travel position, the first scent is discharged, thereby making it possible to restrain drowsiness of a driver while the driver is driving.

In the vehicle of the present disclosure, the first scent may be a scent having a drowsiness prevention effect. This makes is possible to further restrain drowsiness of the driver while the driver is driving.

In the vehicle of the present disclosure, when the shift position is the non-travel position, the control device may control the scent discharge device such that no scent is discharged into the vehicle cabin. This makes it possible to restrain consumption of a scent source in the scent discharge device when the shift position is the non-drive position.

In the vehicle of the present disclosure, when the shift position is the non-travel position, the control device may control the scent discharge device such that a second scent is discharged into the vehicle cabin, the second scent being different from the first scent. In this case, the second scent may be a scent having a relaxation effect. This allows the driver to be relaxed while the driver is not driving.

In the vehicle of the present disclosure, in a case where the shift position has been changed from the non-travel position to the travel position, when a condition that the vehicle has traveled only by a first predetermined distance after the change of the shift position and/or a condition that a first predetermined time has passed after the change of the shift position are established, the control device may control the scent discharge device such that the scent discharge device starts to discharge the scent into the vehicle cabin. With such a configuration, it is possible to restrain consumption of a scent source in the scent discharge device until a condition under which the scent is discharged is established.

In the vehicle of the present disclosure, when a condition that the vehicle has traveled only by a second predetermined distance after start of current scent discharge and/or a condition that a second predetermined time has passed after the start of the current scent discharge are established while the scent discharge device is discharging the scent into the vehicle cabin, the control device may control the scent discharge device such that the scent discharge device changes the scent discharged into the vehicle cabin to a different scent. With such a configuration, it is possible to further restrain drowsiness of the driver while the driver is driving for a long time or the driver is driving a long distance.

In the vehicle of the present disclosure, the scent discharge device may discharge the scent to a driver seat in the vehicle cabin. With such a configuration, it is possible to restrain the scent from being unnecessarily sensed by an occupant other than the driver.

A control device of the present disclosure is a control device provided in a vehicle including a scent discharge device configured to discharge a scent into a vehicle cabin, and the control device is configured to control the scent discharge device. When a shift position of the vehicle is a travel position, the control device controls the scent discharge device such that a first scent is discharged into the vehicle cabin, and when the shift position of the vehicle is a non-travel position, the control device changes the control on the scent discharge device from the control performed when the shift position is the travel position to a different control.

When the shift position of the vehicle is the travel position, the control device of the present disclosure controls the scent discharge device such that the first scent is discharged into the vehicle cabin, and when the shift position of the vehicle is the non-travel position, the control device changes the control on the scent discharge device from the control performed when the shift position is the travel position to a different control. Hereby, when the shift position is the travel position, the first scent is discharged, thereby making it possible to restrain drowsiness of a driver while the driver is driving.

A program of the present disclosure is a program causing a computer to function as a control device configured to control a scent discharge device provided in a vehicle, the scent discharge device being configured to discharge a scent into a vehicle cabin. The program includes a step of, when a shift position is a travel position, controlling the scent discharge device such that a first scent is discharged into the vehicle cabin, and when the shift position is a non-travel position, changing the control on the scent discharge device from the control performed when the shift position is the travel position to a different control.

In the program of the present disclosure, when the shift position is the travel position, the scent discharge device is controlled such that the first scent is discharged into the vehicle cabin, and when the shift position of the vehicle is the non-travel position, the control on the scent discharge device is changed from the control performed when the shift position is the travel position to a different control. Hereby, when the shift position is the travel position, the first scent is discharged, thereby making it possible to restrain drowsiness of a driver while the driver is driving.

### BRIEF DESCRIPTION OF THE DRAWINGS

Features, advantages, and technical and industrial significance of exemplary embodiments of the disclosure will be described below with reference to the accompanying drawings, in which like numerals denote like elements, and wherein:

FIG. 1 is a configuration diagram illustrating an outline of a configuration of an automobile 20 as one embodiment of the present disclosure; and

FIG. 2 is a flowchart illustrating an example of a processing routine executed by an ECU 50.

### DETAILED DESCRIPTION OF EMBODIMENTS

Next will be described a mode for carrying out the disclosure with reference to an embodiment.

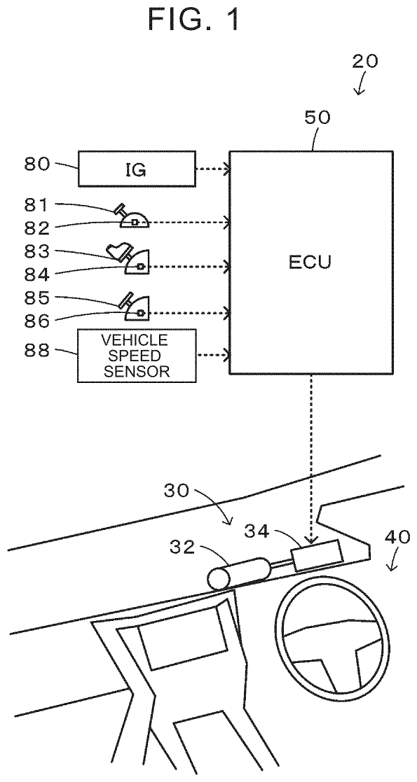

FIG. 1 is a configuration diagram illustrating an outline of a configuration of an automobile 20 as one embodiment of the present disclosure. As illustrated herein, the automobile 20 according to the embodiment includes a scent discharge device 30 and an electronic control unit (hereinafter referred to as “ECU”) 50. The automobile 20 travels based on power from a driving device such as an engine or a motor (not shown).

The scent discharge device 30 includes a plurality of scent cartridges 32 and a blower 34. The scent cartridges 32 are configured, for example, as storage devices in which respective solutions are stored, the solutions containing respective scent sources that generate different scents. The blower 34 is configured as an air-blow device that can send, to a driver seat 40, a scent discharged from each of the scent cartridges 32. Note that the blower 34 may be a blower that is also used as a blower for an air-conditioning device (not shown) configured to perform air-conditioning in a vehicle cabin. The scent discharge device 30 is controlled by the ECU 50 such that the scent cartridge 32 to be used from among the scent cartridges 32 is connected to the blower 34 (the scent cartridge 32 is released to the blower 34 side) so as to discharge a scent to the driver seat 40 by air-blow caused by the blower 34. The scent discharge device 30 can switch the scent cartridge 32 to be used among the scent cartridges 32 in response to a user operation or a control signal from the ECU 50.

Although not illustrated herein, the ECU 50 is configured as a microprocessor mainly constituted by a CPU and includes a ROM in which a processing program is stored, a RAM in which data is stored temporarily, a nonvolatile flash memory, input and output ports, and a communication port, in addition to the CPU. Signals from various sensors are input into the ECU 50 via the input port. Examples of the signals input into the ECU 50 include, for example: an ignition signal from an ignition switch 80; and a shift position SP from a shift position sensor 82 configured to detect an operation position of a shift lever 81. Further, the examples of the signals input into the ECU 50 include: an accelerator pedal depression amount Acc from an accelerator pedal position sensor 84 configured to detect a depression amount of an accelerator pedal 83; a brake depression amount BP from a brake pedal position sensor 86 configured to detect a depression amount of a brake pedal 85; and a vehicle speed V from a vehicle speed sensor 88. Here, as the shift position SP, a parking position (P-position), a reverse position (R-position), a neutral position (N-position), a forward position (D-position), and so on are prepared.

Various control signals are output from the ECU 50 via the output port. Examples of the signals output from the ECU 50 include, for example: a control signal to the driving device such as an engine or a motor (not shown); and a control signal to the scent discharge device 30. The automobile 20 configured as such in the embodiment travels such that the driving device (not shown) is controlled by the ECU 50.

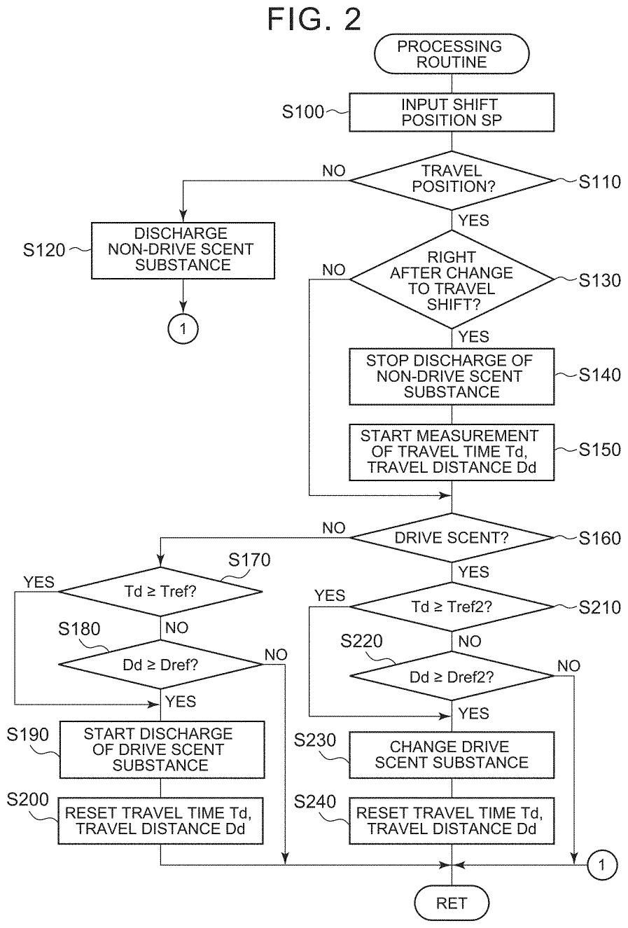

Next will be described the operation of the automobile 20 of the embodiment, particularly, the operation of the scent discharge device 30. FIG. 2 is a flowchart illustrating one example of a processing routine executed by the ECU 50. This routine is executed repeatedly.

When the processing routine of FIG. 2 is executed, the ECU 50 first receives data on the shift position SP from the shift position sensor 82, etc., (step S100) and determines whether or not the shift position SP thus received is a travel position or not (step S110). In this process, when the shift position SP is the D-position for forward travel or the R-position for reverse travel, the ECU 50 determines that the shift position SP is the travel position, and when the shift position SP is a position (e.g., the P-position for parking or the N-position that is neutral) other than the travel position, the ECU 50 determines that the shift position SP is a non-travel position. When the shift position SP is not the travel position (when the shift position SP is the non-travel position), the ECU 50 controls the scent discharge device 30 so that a non-drive scent is discharged (when a drive scent is discharged, the ECU 50 switches the scent to the non-drive scent so that the non-drive scent is discharged) (step S120), and the routine is finished. Here, as the non-drive scent, a scent having a relaxation effect (e.g., lavender, cedar wood, and the like) is used. This allows a driver to be relaxed while the driver is not driving.

When the shift position SP is the travel position in step S110, the ECU 50 determines whether or not the shift position SP is right after a change from the non-travel position to the travel position (step S130). This determination can be performed by checking, for example, a previous shift position SP (previous SP) and a latest shift position SP. When the shift position SP is right after the change from the non-travel position to the travel position, the ECU 50 stops the discharge of the non-drive scent (step S140) and starts measurement of a travel time Td and a travel distance Dd (step S150). Here, the travel time Td indicates a time passing after the shift position SP has changed from the non-travel position to the travel position. The travel distance Dd indicates a travel distance after the shift position SP has changed from the non-travel position to the travel position. When the shift position SP is not right after the change from the non-travel position to the travel position in step S130, the ECU 50 proceeds to step S160 without executing the processes of steps S140 and S150.

Subsequently, the ECU 50 determines whether the drive scent (described later) is being discharged or not (step S160). When the drive scent is not being discharged, the ECU 50 compares the travel time Td with a threshold Tref (step S170) and also compares the travel distance Dd with a threshold Dref (step S180). Here, the thresholds Tref, Dref are thresholds base on which the ECU 50 determines whether the automobile 20 keeps its travelable state to some extent after the shift position SP is changed from the non-travel position to the travel position, and the thresholds Tref, Dref are determined in advance by experiment and analysis. As the threshold Tref, a time of about 30 minutes to 60 minutes is used, for example. As the threshold Dref, a distance corresponding to the time of about 30 minutes to 60 minutes is used, for example. When the travel time Td is less than the threshold Tref and the travel distance Dd is less than the threshold Dref, the routine is finished without discharging the drive scent. This makes it possible to restrain consumption of the scent cartridges 32 (the scent sources stored in the scent cartridges 32) of the scent discharge device 30 until a condition under which the scent is discharged is established.

When the travel time Td is equal to or more than the threshold Tref in step S170, or when the travel distance Dd is equal to or more than the threshold Dref in step S180, the ECU 50 controls the scent discharge device 30 such that the drive scent is discharged (step S190), and the ECU 50 resets the travel time Td and the travel distance Dd (step S200). Then, the routine is finished. Here, as the drive scent, a scent having a drowsiness prevention effect (e.g., eucalyptus, grapefruit, and the like) is used. This makes it possible to restrain drowsiness of the driver while the driver is driving. At this time, a drive scent substance is discharged to the driver seat 40, and therefore, it is possible to restrain the drive scent from being unnecessarily sensed by an occupant other than the driver.

When the drive scent is being discharged in step S160, the ECU 50 compares the travel time Td with a threshold Tref2 (step S210) and also compares the travel distance Dd with a threshold Dref2 (step S220). Here, the thresholds Tref2, Dref2 are thresholds base on which the ECU 50 determines whether or not the automobile 20 keeps its travelable state after the current discharge of the drive scent is started, and the thresholds Tref2, Dref2 are determined in advance by experiment and analysis. As the threshold Tref2, a time of about 30 minutes to 60 minutes is used, for example. As the threshold Dref2, a distance corresponding to the time of about 30 minutes to 60 minutes is used, for example. When the travel time Td is less than the threshold Tref2 and the travel distance Dd is less than the threshold Dref2, the routine is finished without switching the drive scent.

When the travel time Td is equal to or more than the threshold Tref2 in step S210 or when the travel distance Dd is equal to or more than the threshold Dref2 in step

S220, the ECU 50 controls the scent discharge device 30 such that the scent discharge device 30 discharges a scent by changing the drive scent currently discharged to a drive scent of a different type from the drive scent currently discharged (step S230), and then, the ECU 50 resets the travel time Td and the travel distance Dd (step S240). Here, the routine is finished. This makes is possible to further restrain drowsiness of the driver while the driver is driving for a long time or driving a long distance.

In the automobile 20 of the embodiment described above, when the shift position SP is the travel position, the ECU 50 controls the scent discharge device 30 such that the drive scent is discharged to the driver seat 40. Hereby, the drive scent is discharged when the shift position SP is the travel position, thereby making it possible to restrain drowsiness of the driver while the driver is driving.

In the automobile 20 of the embodiment, a scent is discharged to the driver seat 40 by the scent discharge device 30. However, the scent may be discharged to part of the vehicle cabin other than the driver seat 40 instead of or in addition to the driver seat 40.

Further, only in a case where the non-drive scent is discharged, the scent may be discharged to part of the vehicle cabin other than the driver seat 40 instead of or in addition to the driver seat 40. With such a configuration, when the shift position SP is the non-travel position, occupants in the vehicle cabin can be relaxed by the non-drive scent.

In the automobile 20 of the embodiment, when the travel time Td is equal to or more than the threshold Tref or when the travel distance Dd is equal to or more than the threshold Dref, discharge of the drive scent is started. However, the drive scent may be discharged when the travel time Td is equal to or more than the threshold Tref and the travel distance Dd is equal to or more than the threshold Dref. Further, the drive scent may be discharged regardless of the travel time Td and the travel distance Dd.

In the automobile 20 of the embodiment, when the travel time Td is equal to or more than the threshold Tref2 or when the travel distance Dd is equal to or more than the threshold Dref2, the drive scent currently discharged is changed to a drive scent of a different type from the drive scent currently discharged, and the drive scent of the different type is discharged. However, when the travel time Td is equal to or more than the threshold

Tref2 and the travel distance Dd is equal to or more than the threshold Dref2, the drive scent currently discharged may be changed to a drive scent of a different type from the drive scent currently discharged so that the drive scent of the different type is discharged. Further, the drive scent that has been already discharged may not be changed regardless of the travel time Td and the travel distance Dd.

In the automobile 20 of the embodiment, when the shift position SP is the non-travel position, the non-drive scent is discharged. However, no scent may be discharged. With such a configuration, when the shift position SP is the non-travel position, it is possible to restrain consumption of the scent cartridges 32 (the scent sources stored in the scent cartridge 32) of the scent discharge device 30.

In the embodiment, the present disclosure is applied to the form of the automobile 20. However, the present disclosure may be applied to the form of the ECU 50 as a control device to be provided in the automobile 20 or the form of a program that causes the ECU 50 to function as a control device for the scent discharge device 30.

The following describes a correspondence between main elements of the embodiment and main elements of the disclosure described in the field of SUMMARY. In the present embodiment, the scent discharge device 30 corresponds to a “scent discharge device,” and the ECU 50 corresponds to a “control device.” Further, the scent cartridge 32 corresponds to a “storage portion” and a “controlling portion.”

Note that, since the embodiment is an example to specifically describe a mode for carrying out the disclosure described in the field of SUMMARY, the correspondence between the main elements of the embodiment and the main elements of the disclosure described in the field of SUMMARY does not limit the elements of the disclosure described in the field of SUMMARY. That is, the disclosure described in the field of

SUMMARY should be interpreted based on the description of the field, and the embodiment is just a concrete example of the disclosure described in the field of SUMMARY.

The mode for carrying out the disclosure has been described with reference to the embodiment, but it is needless to say that the present disclosure is not limited to the above embodiment at all and may be performable in various embodiments as long as the various embodiments are not beyond the gist thereof.

The present disclosure is usable in manufacture industries or the like of a vehicle, a control device, and a program.

What is claimed is:

1. A vehicle comprising:
a scent discharge device configured to discharge a scent into a vehicle cabin; and
a control device configured to control the scent discharge device, wherein when a shift position is a travel position, the control device controls the scent discharge device such that a first scent is discharged into the vehicle cabin, and when the shift position is a non-travel position, the control device changes the control on the scent discharge device from the control performed when the shift position is the travel position to a different control.

2. The vehicle according to claim 1, wherein the first scent is a scent having a drowsiness prevention effect.

3. The vehicle according to claim 1, wherein, when the shift position is the non-travel position, the control device controls the scent discharge device such that no scent is discharged into the vehicle cabin.

4. The vehicle according to claim 1, wherein, when the shift position is the non-travel position, the control device controls the scent discharge device such that a second scent is discharged into the vehicle cabin, the second scent being different from the first scent.

5. The vehicle according to claim 4, wherein the second scent is a scent having a relaxation effect.

6. The vehicle according to claim 1, wherein, in a case where the shift position has been changed from the non-travel position to the travel position, when a condition that the vehicle has traveled only by a first predetermined distance after the change of the shift position and/or a condition that a first predetermined time has passed after the change of the shift position are established, the control device controls the scent discharge device such that the scent discharge device starts to discharge the scent into the vehicle cabin.

7. The vehicle according to claim 1, wherein, when a condition that the vehicle has traveled only by a second predetermined distance after start of current scent discharge and/or a condition that a second predetermined time has passed after the start of the current scent discharge are established while the scent discharge device is discharging the scent into the vehicle cabin the control device controls the scent discharge device such that the scent discharge device changes the scent discharged into the vehicle cabin to a different scent.

8. The vehicle according to claim 1, wherein the scent discharge device discharges the scent to a driver seat in the vehicle cabin.

9. A control device provided in a vehicle including a scent discharge device configured to discharge a scent into a vehicle cabin, the control device being configured to control the scent discharge device, wherein, when a shift position of the vehicle is a travel position, the control device controls the scent discharge device such that a first scent is discharged into the vehicle cabin, and when the shift position of the vehicle is a non-travel position, the control device changes the control on the scent discharge device from the control performed when the shift position is the travel position to a different control.

10. A program causing a computer to function as a control device configured to control a scent discharge device provided in a vehicle, the scent discharge device being configured to discharge a scent into a vehicle cabin, the program comprising a step of, when a shift position is a travel position, controlling the scent discharge device such that a first scent is discharged into the vehicle cabin, and when the shift position is a non-travel position, changing the control on the scent discharge device from the control performed when the shift position is the travel position to a different control.
